(19) United States
(12) Patent Application Publication
(10) Pub. No.: US 2019/0213352 A1
(43) Pub. Date: Jul. 11, 2019

(54) SYSTEMS AND METHODS FOR SECURING REDUCED PRESSURE THERAPY DEVICES

(71) Applicant: Smith & Nephew, Inc., Memphis, TN (US)

(72) Inventors: Billy K. Rios, Half Moon Bay, CA (US); Felix C. Quintanar, Hull (GB)

(21) Appl. No.: 16/333,948

(22) PCT Filed: Sep. 27, 2017
(86) PCT No.: PCT/US2017/053839

§ 371 Date: Mar. 15, 2019

Related U.S. Application Data
(60) Provisional application No. 62/401,019, filed on Sep. 28, 2016.

(51) Int. Cl. G06F 21/62; A61M 1/00; H04L 9/32; H04L 29/06; H04W 12/06; G16H 20/30; G16H 40/67

(52) U.S. Cl. CPC G06F 21/6245; A61M 2205/52; H04L 9/3263; H04L 63/0869; H04L 9/3273; H04W 12/06; H04L 63/0823; H04L 63/0428; G16H 20/30; G16H 40/67; H04L 2209/88; A61M 2205/3553; A61M 2205/6009; H04L 63/164; A61M 1/0088

(57) ABSTRACT

Embodiments of negative pressure wound therapy devices, systems and methods are disclosed. In some embodiments, a negative pressure wound therapy apparatus includes network connection capabilities allowing the TNP apparatus to transmit data via a communications network. The negative pressure wound therapy apparatus can include security measures to prevent exposure to security risks associated with the network connection.

### CROSS-REFERENCE TO RELATED APPLICATIONS

This application claims the benefit of U.S. Provisional Application No. 62/401,019, filed Sep. 28, 2016, the disclosure of which is hereby incorporated by reference in its entirety.

### BACKGROUND

### Field

Embodiments of the present disclosure relate to apparatuses, systems, and methods for the treatment of wounds, for example using dressings in combination with negative pressure wound therapy, or non-negative pressure wound therapy.

### Description of the Related Art

Many different types of wound dressings are known for aiding in the healing process of a human or animal. These different types of wound dressings include many different types of materials and layers, for example, gauze, pads, foam pads or multi-layer wound dressings. Topical negative pressure therapy, sometimes referred to as vacuum assisted closure, negative pressure wound therapy, or reduced pressure wound therapy, is widely recognized as a beneficial mechanism for improving the healing rate of a wound. Such therapy is applicable to a broad range of wounds such as incisional wounds, open wounds and abdominal wounds or the like.

However, prior art negative pressure wound therapy or other wound therapy systems provide little security measures to prevent exposure to security risks. This can cause the negative pressure therapy system to be exposed to security concerns.

### SUMMARY

Disclosed embodiments relate to negative pressure wound closure devices, methods, and systems that facilitate closure of a wound. It will be understood by one of skill in the art that the wounds described herein this specification may encompass any wound, and are not limited to a particular location or type of wound. The devices, methods, and systems may operate to reduce the need for repetitive replacement of wound dressing material currently employed and can advance the rate of healing. The devices, methods, and systems may be simultaneously used with negative pressure to remove wound fluids.

In certain embodiments, an apparatus for applying negative pressure to a wound is provided, the apparatus comprises a controller coupled to a memory and a negative pressure source configured to provide negative pressure via a fluid flow path to a wound covered by a wound dressing. The controller may be configured to operate the negative pressure source to provide negative pressure to the wound, communicate with a remote computing device via a computer network according to a security protocol, and/or process data received from the remote computing device according to a security rule. The security protocol can comprise periodically assigning a new IP address to the apparatus.

The apparatus of the preceding paragraph can further comprise one or more of the following features: The security protocol can further comprise assigning a new IP address to the apparatus for each communication request to the remote computing device and encrypting communications with the remote computing device through mutual authentication. The security rule can comprise not responding to any redirect requests to a network address different from a network address of the remote computing device. The mutual authentication may be performed via security certificates stored in the memory of the apparatus and on the remote computing device. The security certificate stored in the memory can uniquely identify the apparatus. The memory can stores instructions that, when executed by the controller, cause the controller to operate the negative pressure source, communicate with the remote computing device, and process data received from the remote computing device. The security rule may comprise, in response to receiving from the computing device an update of at least some instructions stored in the memory, verifying an identity of an author of the update prior to updating the at least some instructions. The apparatus may further comprise one or more anti-tampering mechanisms configured to indicate unauthorized use of the apparatus. The controller may be configured to process the data according to the security rule so that access to the data provided by the controller via the computer network is limited to one or more authenticated devices. The controller may be configured to receive the data according to the security rule so that the data is enabled to adjust a first function performable by the controller and prevented from adjusting a second function performable by the controller. The security rule can comprise enforcing code signing. The security rule can comprise enforcing transport encryption. Transport encryption may utilize transport layer security. The security rule can comprise utilizing mutual authentication. Mutual authentication may be established through use of server and client certificates. The data can comprise data indicative of operations of the negative pressure source or identification information for a user of the negative pressure source. The data can comprise data indicative of operations of the negative pressure source or patient data for a user of the apparatus.

In certain embodiments, a method for operating a negative pressure wound therapy apparatus, the method comprises: activating a negative pressure source configured to provide negative pressure via a fluid flow path to a wound covered by a wound dressing, communicating with a remote computing device via a computer network according to a security protocol, and processing data received from the remote computing device according to a security rule. The security protocol can comprise periodically assigning a new IP address to the apparatus. The method may be performed by a controller of the apparatus.

The method of the preceding paragraph can further include one or more of the following features: The security protocol can further comprise assigning a new IP address to the apparatus for each communication request to the remote computing device and encrypting communications with the remote computing device through mutual authentication. The security rule can comprise not responding to any redirect requests to a network address different from a network address of the remote computing device. Mutual authentication may be performed via security certificates stored in a memory of the apparatus and on the remote computing device. The security certificate may be stored in the memory uniquely identifies the apparatus. The memory may store instructions that, when executed by the controller, cause the controller to operate the negative pressure source, communicate with the remote computing device, and process data received from the remote computing device. The security rule can comprise, in response to receiving from the computing device an update of at least some instructions stored in the memory, verifying an identity of an author of the update prior to updating the at least some instructions. The apparatus can further comprise one or more anti-tampering mechanisms configured to indicate unauthorized use of the apparatus. The controller may be configured to process the data according to the security rule so that access to the data provided by the controller via the computer network is limited to one or more authenticated devices. The controller may be configured to receive the data according to the security rule so that the data is enabled to adjust a first function performable by the controller and prevented from adjusting a second function performable by the controller. The security rule can comprise enforcing code signing. The security rule can comprise enforcing transport encryption. Transport encryption may utilize transport layer security. The security rule can comprise utilizing mutual authentication. Mutual authentication may be established through use of server and client certificates. The data can comprise data indicative of operations of the negative pressure source or identification information for a user of the negative pressure source. The data can comprise data indicative of operations of the negative pressure source or patient data for a user of the apparatus.

Other embodiments of wound closure devices, stabilizing structures and associated apparatuses are described below.

### BRIEF DESCRIPTION OF THE DRAWINGS

Embodiments of the present disclosure will now be described hereinafter, by way of example only, with reference to the accompanying drawings in which:

FIG. 1 illustrates a negative pressure therapy system according to some embodiments;

FIG. 2 illustrates a negative pressure therapy system according to some embodiments;

FIG. 3 illustrates a negative pressure therapy system according to some embodiments;

FIG. 4 illustrates a negative pressure therapy system according to some embodiments;

FIG. 5 illustrates a negative pressure therapy system according to some embodiments;

FIG. 6 illustrates a negative pressure therapy system according to some embodiments; and

FIG. 7 illustrates a negative pressure therapy system according to some embodiments.

### DETAILED DESCRIPTION

### Overview

Embodiments disclosed in this section or elsewhere in this specification relate to apparatuses and methods of treating a wound with reduced pressure, including pump and wound dressing components and apparatuses. The apparatuses and components comprising the wound overlay and packing materials, if any, are sometimes collectively referred to in this section or elsewhere in this specification as dressings.

It will be appreciated that throughout this specification reference is made to a wound. It is to be understood that the term wound is to be broadly construed and encompasses open and closed wounds in which skin is torn, cut or punctured or where trauma causes a contusion, or any other superficial or other conditions or imperfections on the skin of a patient or otherwise that benefit from reduced pressure treatment. A wound is thus broadly defined as any damaged region of tissue where fluid may or may not be produced. Examples of such wounds include, but are not limited to, abdominal wounds or other large or incisional wounds, either as a result of surgery, trauma, sterniotomies, fasciotomies, or other conditions, dehisced wounds, acute wounds, chronic wounds, subacute and dehisced wounds, traumatic wounds, flaps and skin grafts, lacerations, abrasions, contusions, burns, electrical burns, diabetic ulcers, pressure ulcers, stoma, surgical wounds, trauma and venous ulcers or the like.

Embodiments of the present disclosure are generally applicable to use in topical negative pressure (TNP) or reduced pressure therapy systems. Briefly, negative pressure wound therapy assists in the closure and healing of many forms of “hard to heal” wounds by reducing tissue edema, encouraging blood flow and granular tissue formation, or removing excess exudate and can reduce bacterial load (and thus infection risk). In addition, the therapy allows for less disturbance of a wound leading to more rapid healing. TNP therapy systems can also assist in the healing of surgically closed wounds by removing fluid. In some embodiments, TNP therapy helps to stabilize the tissue in the apposed position of closure. A further beneficial use of TNP therapy can be found in grafts and flaps where removal of excess fluid is important and close proximity of the graft to tissue is required in order to ensure tissue viability.

Various example embodiments and features related to methods and apparatuses for dressing and treating a wound with reduced pressure therapy or TNP therapy are described and/or contemplated within International Patent Application No. PCT/US2014/026692 (herein referred to as the '692 Application), incorporated herein by reference in its entirety. The embodiments described below are compatible with and can be part of the embodiments described in the '692 Application, and some or all of the features described below can be used or otherwise combined with any of the features described in the '692 Application.

In some embodiments, a TNP apparatus may contain network connection capabilities allowing the TNP apparatus to transmit data via a communications network, such as a cellular network. The communications network can, for instance, provide access to the Internet or additional device functionality to the TNP apparatus. The TNP apparatus may include security measures to prevent exposure to security risks associated with network connection capabilities. As such, the security measures may be incorporated into the TNP apparatus or the negative pressure therapy system in which the TNP apparatus communicates to limit exposure of the TNP apparatus or the negative pressure therapy system to security concerns.

### Reduced Pressure Therapy Systems and Methods

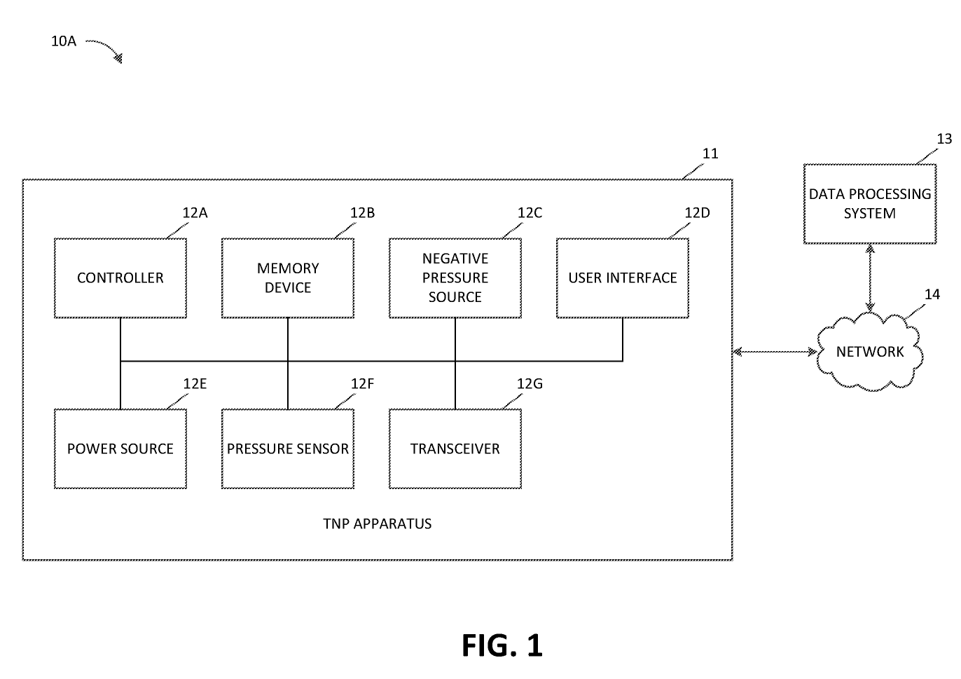

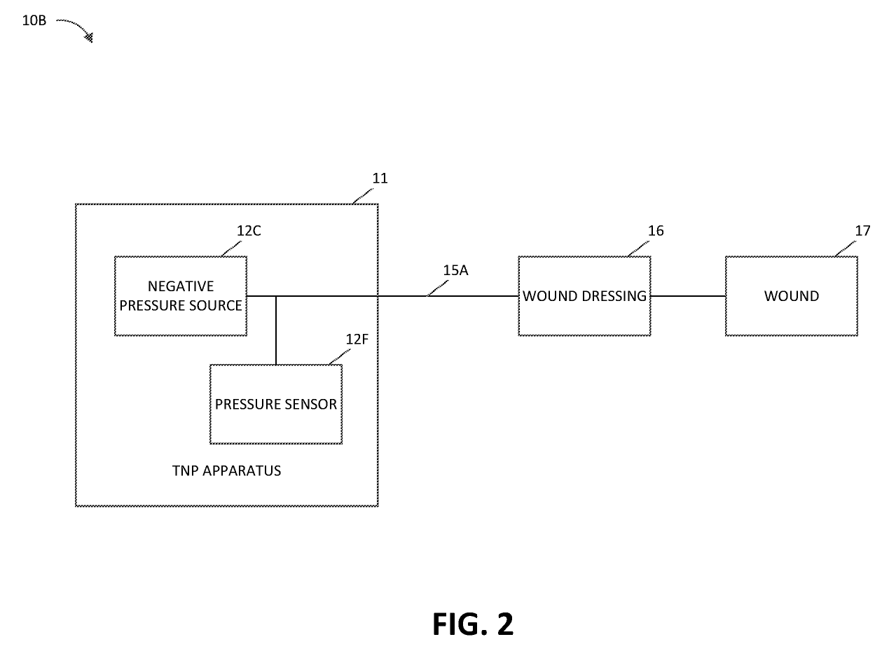

FIG. 1 illustrates a negative pressure therapy system 10A that includes a TNP apparatus 11 and a data processing system 13. The TNP apparatus 11 can be used to treat a wound using a wound dressing that is in fluidic communication with the TNP apparatus 11 via a fluid flow path. The TNP apparatus 11 can include a controller 12A, a memory device 12B, a negative pressure source 12C, a user interface 12D, a power source 12E, a pressure sensor 12F, and a transceiver 12G that are configured to electrically communicate with one another. The power source 12E can provide power to one or more components of the TNP apparatus 11. The TNP apparatus 11 can operate at the pressure levels and using control approaches similar to those described in the '692 Application, but may differ, at least in some instances, as described herein. In some implementations, the TNP apparatus 11 can configured the same as or similarly to the pump assembly 150 of FIG. 1, the pump assembly 230 of FIGS. 2A-F, or the pump assembly 1520 of FIG. 15 in the '692 Application, and the data processing system 13 can be configured at least partially the same as or similarly to the remote computer 1540 of FIG. 15 in the '692 Application.

The controller 12A can control operations of one or more other components of the TNP apparatus 11 according at least to instructions stored in the memory device 12B. The controller 12A can, for instance, control operations of and supply of negative pressure by the negative pressure source 12C. The negative pressure source 12C can include a pump, such as, without limitation, a rotary diaphragm pump or other diaphragm pump, a piezoelectric pump, a peristaltic pump, a piston pump, a rotary vane pump, a liquid ring pump, a scroll pump, a diaphragm pump operated by a piezoelectric transducer, or any other suitable pump or micropump or any combinations of the foregoing. The user interface 12D can include one or more elements that receive user inputs or provide user outputs to a patient or caregiver. The one or more elements that receive user inputs can include buttons, switches, dials, touch screens, or the like.

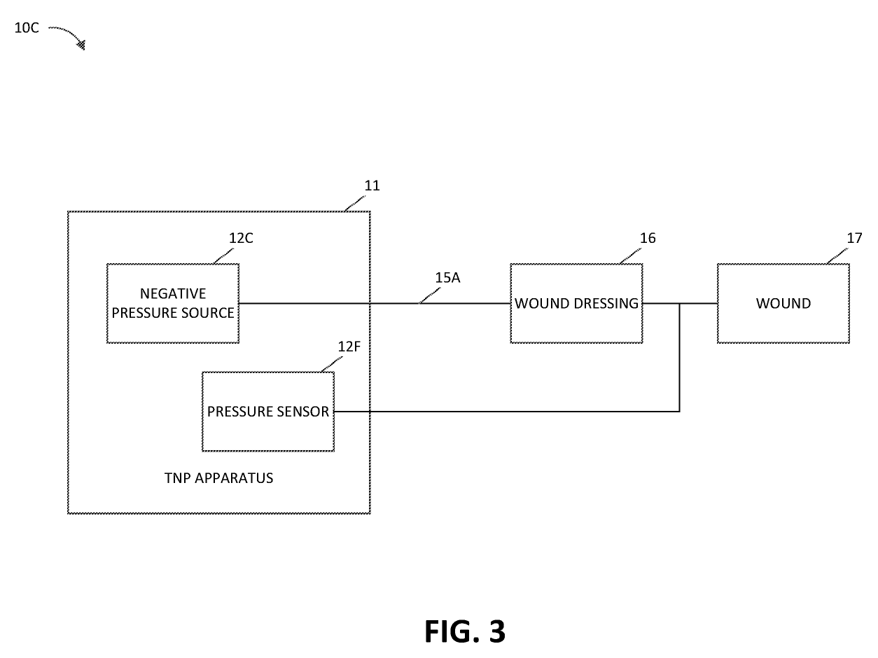

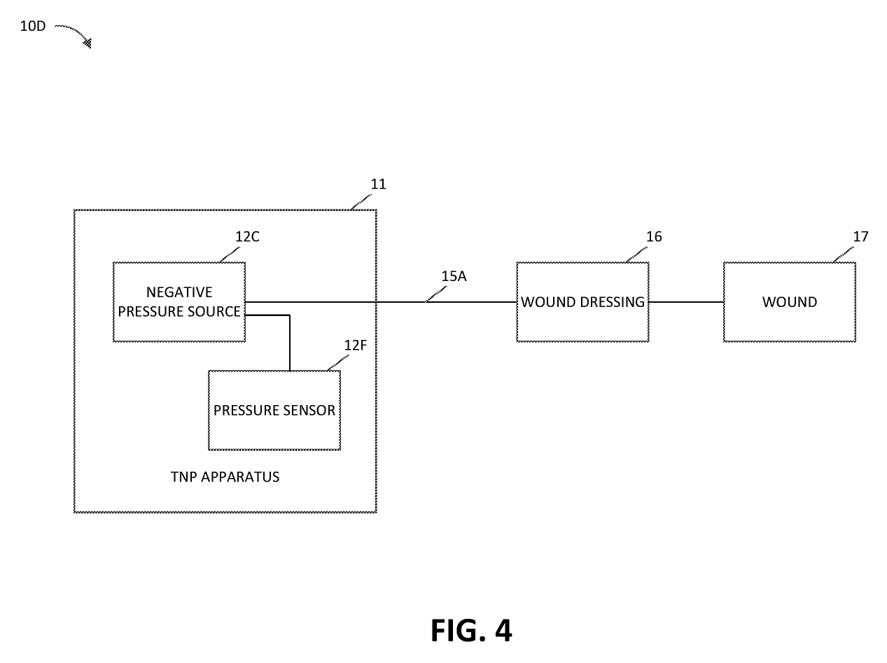

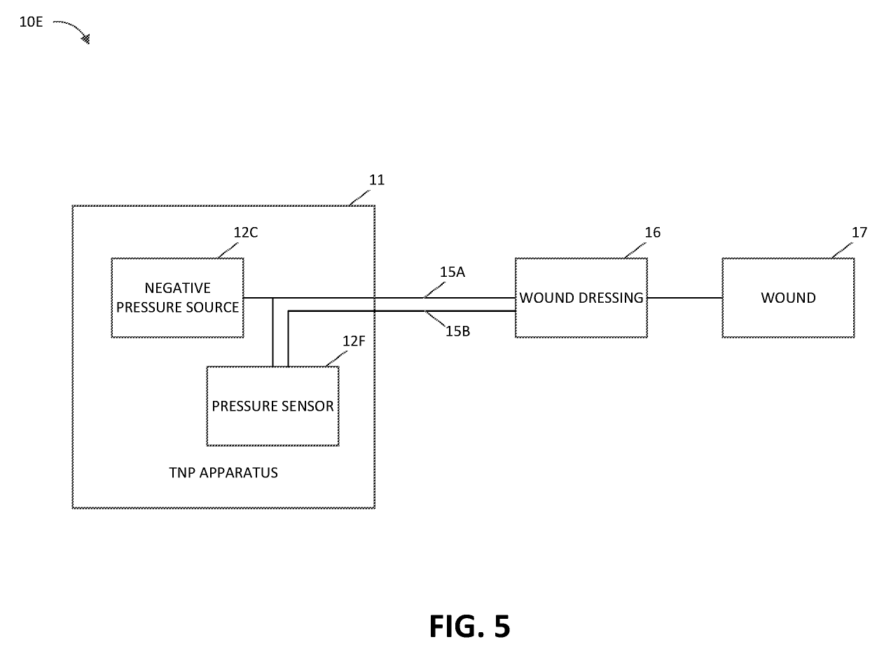

The pressure sensor 12F can be used to monitor pressure underneath a wound dressing, such as (i) pressure in a fluid flow path connecting the negative pressure source 12C and the wound dressing as illustrated by FIG. 2, (ii) pressure at the wound dressing as illustrated by FIG. 3, or (iii) pressure at or in the negative pressure source 12C as illustrated by FIG. 4. In some implementations, the pressure sensor 12F can include at least two pressure sensors that are positioned in or fluidically connected to the fluid flow path to permit differential measurement of the pressure, such as illustrated by FIG. 5. For example, a first pressure sensor can be positioned upstream of the wound (such as at or near the inlet of the negative pressure source 12C) and a second pressure sensor can be positioned to detect pressure at or near the wound or at or near a canister. This configuration can be accomplished by incorporating, in addition to one or more lumens forming a first fluid flow path connecting the negative pressure source 12C to the wound, a second fluid flow path that includes one or more lumens connecting the TNP apparatus 11 to the wound and through which the second pressure sensor can monitor pressure at or near the wound or at or near a canister. The first and second fluid flow paths can be fluidically isolated from each other. When the at least two pressure sensors are used, the rate of change of pressure (for example, in peak-to-peak pressure or maximum pressure, in the first and second fluid flow paths can be determined and the difference in pressure detected between the first and second pressure sensors can be determined. These values can be used separately or together to detect various operational conditions, such as leaks, blockages, canister full, presence of blood in the first fluid flow path or the second fluid flow path, etc. In some implementations, multiple redundant pressure sensors can be provided to protect against failure of one or more of the pressure sensors.

The transceiver 12G can be used to communicate with the data processing system 13 via a network 14. The transceiver 12G can, for example, transmit device usage data like alarms, measured pressure, or changes to a therapy program administered by the TNP apparatus 11 to the data processing system 13. The network 14 can be a communication network, such as a wireless communications network like a cellular communications network. The memory device 12B can be used to store the device usage data that may be transmitted by the transceiver 12G. The data processing system 13 can, in some implementations, analyze pressure data received from the transceiver 12G to determine whether the received pressure data is indicative of the negative pressure source 12C being in use on a patient, such as using analysis approaches as described with respect to the TNP apparatus 11.

FIG. 2 illustrates a negative pressure therapy system 10B that includes the TNP apparatus 11 of FIG. 1, as well as a first fluid flow path 15A, a wound dressing 16, and a wound 17. The TNP apparatus 11 can be used to treat the wound 17 using the wound dressing 16 that is in fluidic communication with the negative pressure source 12C via the first fluid flow path 15A. In particular, FIG. 2 depicts that the pressure sensor 12F can be positioned in the first fluid flow path 15A, such as at or near an inlet of the TNP apparatus 11, to measure pressure in the first fluid flow path 15A.

FIG. 3 illustrates a negative pressure therapy system 10C that differs from the negative pressure therapy system 10B in that the pressure sensor 12F can instead be positioned to measure pressure at or near the wound dressing 16, such as pressure underneath the wound dressing 16 when the wound dressing 16 is coupled to the wound 17.

FIG. 4 illustrates a negative pressure therapy system 10D that differs from the negative pressure therapy system 10B in that the pressure sensor 12F can instead be positioned to measure pressure at the negative pressure source 12C. In one example, the pressure sensor 12F can be a part of and within the negative pressure source 12C to measure pressure generated by the negative pressure source 12C. In another example, the pressure sensor 12F can be separate from the negative pressure source 12C and positioned to measure pressure at or near an inlet of the negative pressure source 12C.

FIG. 5 illustrates a negative pressure therapy system 10E that differs from the negative pressure therapy system 10B in that the negative pressure therapy system 10E further includes a second fluid flow path 15B, and the pressure sensor 12F can be a differential pressure sensor or include two pressure sensors. If the pressure sensor 12F may include the two pressure sensors, one of the two pressure sensors of the pressure sensor 12F can be positioned in the first fluid flow path 15A to measure pressure in the first fluid flow path 15A, and the other of the two pressure sensors the pressure sensor 12F can be positioned in the second fluid flow path 15B to measure pressure in the second fluid flow path 15B. If the pressure sensor 12F may be the differential pressure sensor, the pressure sensor 12F can be fluidically connected to the first fluid flow path 15A and the second fluid flow path 15B. The first fluid flow path 15A can thus be used by the negative pressure source 12C to provide negative pressure to the wound dressing 16, and the second fluid flow path 15B can be used primarily by the pressure sensor 12F to measure pressure at or near the wound dressing 16, such as under the wound dressing 16. The pressure sensor 12F can thereby be used by the TNP apparatus 11 to perform differential measurement of pressure between pressure supplied by the negative pressure source 12C and pressure at or near the wound dressing 16.

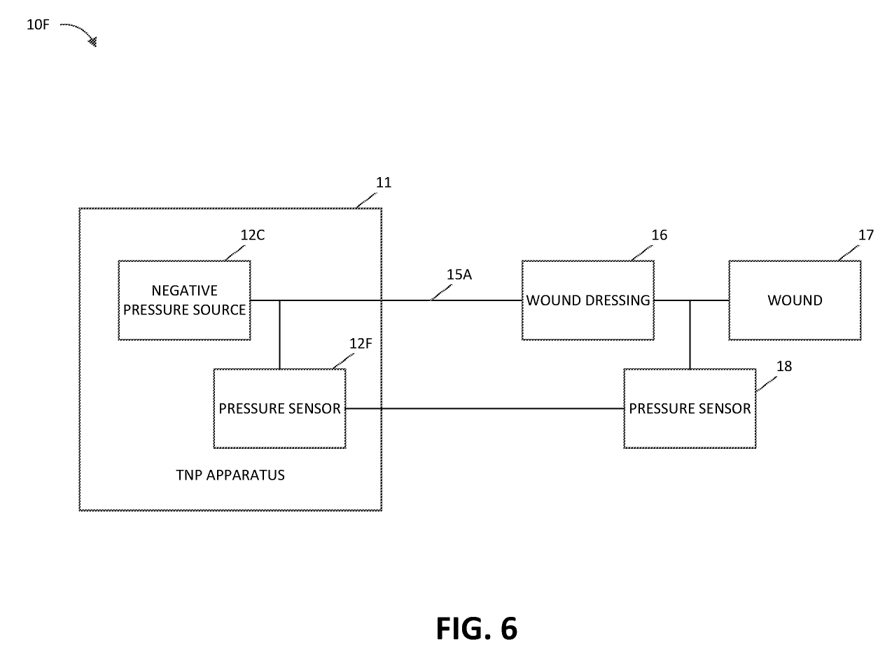

FIG. 6 illustrates a negative pressure therapy system 10F that differs from the negative pressure therapy system 10B in that the negative pressure therapy system 10F can further include an additional pressure sensor 18 positioned to measure pressure at or near the wound dressing 16, such as pressure underneath the wound dressing 16 when the wound dressing 16 is coupled to the wound 17. The additional pressure sensor 18 can generate and output a signal to the TNP apparatus 11 responsive to the pressure measured at the wound dressing 16. The pressure sensor 12F and the additional pressure sensor 18 can thus be used by the TNP apparatus 11 to perform differential measurement of pressure between pressure supplied by the negative pressure source 12C and pressure at or near the wound dressing 16.

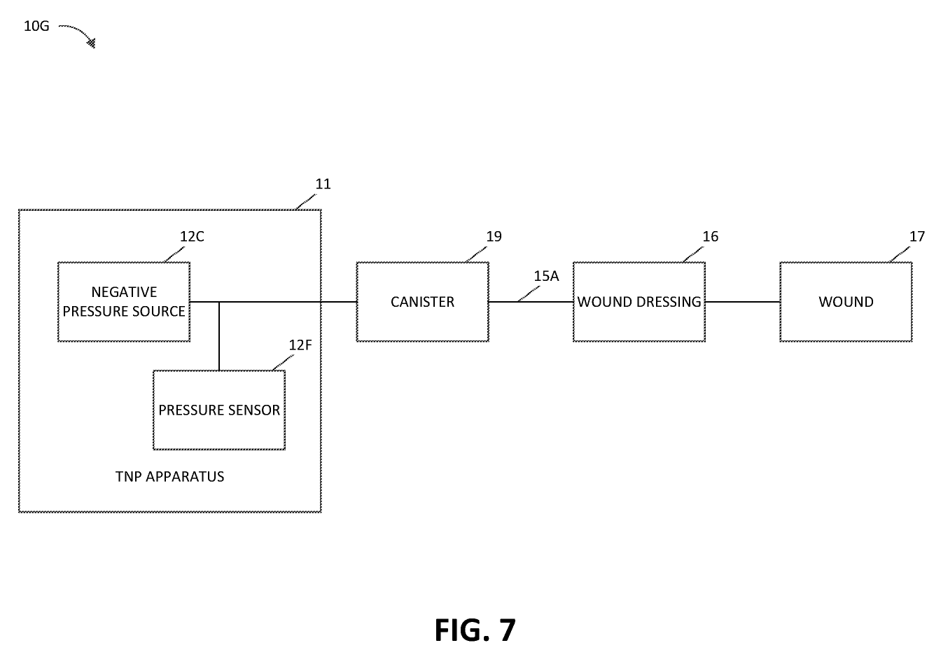

FIG. 7 illustrates a negative pressure therapy system 10G that differs from the negative pressure therapy system 10B in that a canister 19 can be coupled between the negative pressure source 12C and the wound dressing 16 in the first fluid flow path 15A. The canister 19 can collect exudate removed from the wound 17. The examples of FIGS. 3 to 6 can be similarly modified to also include the canister 19, in some implementations.

### Network Connection Capabilities and Security

The TNP apparatus 11 may contain network connection capabilities, such as via the transceiver 12G, allowing the TNP apparatus 11 to transmit data via a communications network, such as a cellular network. The communications network can provide access to the Internet. In some instances, when a HTTP request is made from the TNP apparatus 11 to the data processing system 13, which can be a cloud service in some instances, the TNP apparatus 11 is temporarily assigned an IP address. The TNP apparatus 11 address IP may change for each request made by the apparatus 11. In some instances, the TNP apparatus 11 may initiate communication with other devices and may not accept incoming requests from other devices, such as via the Internet.

Once the TNP apparatus 11 has established a connection interface, such as a cellular and TCP/IP backend, the TNP apparatus 11 may make various HTTP requests to the data processing system 13. For example, health or diagnostic information about the negative pressure therapy system 10A may be passed from or to the TNP apparatus 11 to the data processing system 13. Additionally, patient or therapy data may be transmitted from the TNP apparatus 11 to the data processing system 13. The data processing system 13 may respond with various responses based on processing of received data. In some instances, the various responses utilize a simple response including a minimalistic set of HTTP headers. The negative pressure therapy system 10A may not conduct extensive parsing of data processing system responses. For example, if the data processing system 13 responds with a HTTP 302 redirect, the TNP apparatus 11 may not follow the redirect message to a new URL.

The TNP apparatus 11 can be configured so that software updates may be performed via physical access to a USB or Serial port located on the TNP apparatus 11. Once physical access to the TNP apparatus 11 is established, technicians may utilize custom software to update a motor controller firmware and the apparatus interface software. In some instances, operating system and bootloader updates may be accomplished via a separate process which involves a case of the TNP apparatus 11 being fully opened. The operating system updates may utilize a different set of hardware targeted commercial software.

The software updates to the TNP apparatus 11 may use code signing. Code signing enables verification of the identity of the author of a particular piece of software and provide a means to help ensure the software has not been tampered. Enforcing code signing for uploaded firmware can ensure that software updates developed by the manufacturer of the TNP apparatus 11 may be loaded while software from other providers may not be loaded. Implementing code signing for the TNP apparatus 11 software can span multiple software development lifecycles.

In some instances, the manufacturer of the TNP apparatus 11 or the data processing system 13 may provide “known good” hashes to a third party validation service. Third party validation services can help detect tampered firmware or software during investigations or system inspections. Third party validation services can also provide a mechanism for third party integrity and forensics validation.

In some instances, a data processing system's uniform resource locators (URLs) may be limited to “https://.” In alternative instances, the data processing system URL may be pointed to an arbitrary URL. However, allowing for arbitrary URLs to be passed to the negative pressure therapy system 10A components can allow for arbitrary code execution on the software executing the arbitrary URL.

The name and serial number assigned to the TNP apparatus 11 may be restricted to alphanumeric characters. To accomplish this, the TNP apparatus 11 or the data processing system 13 may incorporate validation checks which use alphanumeric characters for the TNP apparatus 11 serial number and name. Setting the serial number and name for the TNP apparatus 11 to alphanumeric characters can help to avoid security vulnerabilities that may be introduced if other external services assume that the serial number for the TNP apparatus 11 includes alphanumeric characters and consume a non-alphanumeric character value. In alternative instances, the name and serial number of the TNP apparatus 11 may not contain the alphanumeric character restriction.

The TNP apparatus 11 or the data processing system 13 may enforce transport encryption via transport layer security (TLS), for example https://, for data processing system connections. TLS can provide an encrypted tunnel for data to traverse through, thus providing protection against data tampering and data observation, confidentiality of data while in transit over the network, integrity verification of transmitted or received data, and endpoint verification. The Certificate Authority for the transport encryption certificate may be loaded on the TNP apparatus 11 and certificates may also be loaded on the data processing system 13.

The TNP apparatus 11 may be configured to so that its access to web server management consoles is restricted in some implementations. Access to web management interfaces may include access to the data processing system 13. Generally, users of the TNP apparatus 11 may not be expected to access web management interfaces. As such, the management interfaces can be disabled in some instances.

Connection to the data processing system 13 may utilize mutual authentication in some instances. Mutual authentication may prevent unauthorized entities from communicating with the data processing system 13. Additionally, if the TNP apparatus 11 is ever inadvertently connected to a communications networks, such as the Internet, and reachable via TCP/IP, mutual authentication may prevent an alternative device that is not associated with the TNP apparatus 11 or the data processing system 13 from interacting with the TNP apparatus 11 or the data processing system 13. A common form of mutual authentication is provided via certificates placed on both the TNP apparatus 11 and the data processing system 13. Traffic between the TNP apparatus 11 and the data processing system 13 may be “tunneled” through the established, secure connection provided by the mutual authentication. Generally, the mutual authentication can help ensure that systems developed by the manufacturer may communication with the data processing system 13 and not with other devices.

Mutual authentication can be established through the use of server and client certificates. To implement mutual authentication, transport encryption can first be in place, as discussed above. Once transport encryption is in place, client side authentication certificates (typically PKCS12) may be incorporated within the TNP apparatus 11 itself. The client side certificates can be created from the same Certificate Authority used by the transport encryption certificates. The TNP apparatus 11 may have a unique client side certificate, which allows the system's manufacturer to uniquely identify the TNP apparatus 11 when communicating to the data processing system 13. In some instances, a revocation mechanism may be in place to revoke tampered and stolen certificates which might be used to communicate with the data processing system 13.

A file firmware file uploaded to the TNP apparatus 11 may be a compressed representation of the system's file system. Once the compressed file is uploaded, the TNP apparatus 11 may reboot, decompress the file, check each file within the file system for changes, verify the files against a list of MD5 checksums, and then install the file to the appropriate location.

In some instances, several portions of the update process described herein may be circumvented by monitoring update files for path traversal issues, including files with directory traversal strings within the file name. For example, if a directory traversal string is included in a file name within the compressed firmware file, this file can be written to an arbitrary location on the TNP apparatus 11. This process may occur before validation of MD5 checksums occurs and may allow a remote user to place files in locations not normally allowed by a file system update.

The TNP apparatus 11 or the data processing system 13 can be configured to perform obfuscation of data. The TNP apparatus 11 or data processing system 13 may contain sensitive information (such as passwords, proprietary logic, or keys), and these values may be easily extracted from the TNP apparatus 11 without additional security measures. This can make it more difficult to reverse engineer and identify vulnerable segments of the software of the TNP apparatus 11 or the data processing system 13.

The TNP apparatus 11 may include anti-tamper mechanisms to prevent unauthorized personnel from accessing the internals associated with the TNP apparatus 11. These anti-tamper mechanisms can be tamper seals which can provide technicians with an indication that an unauthorized entity has tampered the TNP apparatus 11. Additional or alternative mechanisms can include the usage of technologies, such as eFuse, which separates sensitive device logic from traditional components and makes it difficult to extract information from the TNP apparatus 11. For example, anti-tamper mechanisms that may be incorporated into the TNP apparatus 11 include tamper proof security nuts, bolts, and fasteners; anti-tamper adhesive and seals; cutting or limiting debugging interfaces; antiFuse, and any combination thereof.

### Example Embodiments

1. An apparatus for applying negative pressure to a wound, comprising:

a negative pressure source configured to provide negative pressure via a fluid flow path to a wound dressing; and

a controller configured to:

operate the negative pressure source to provide negative pressure to the wound dressing, and

process data communicated via a computer network according to a security rule.

2. The apparatus of any one or more preceding embodiments, wherein the controller is configured to process the data according to the security rule so that access to the data provided by the controller via the computer network is limited to one or more authenticated devices.

3. The apparatus of any one or more preceding embodiments, wherein the controller is configured to receive the data according to the security rule so that the data is enabled to adjust a first function performable by the controller and prevented from adjusting a second function performable by the controller.

4. The apparatus of any one or more preceding embodiments, wherein the data comprises data indicative of operations of the negative pressure source or identification information for a user of the negative pressure source.

5. A method of operating, using, or manufacturing the apparatus of any preceding embodiment.

### Other Variations

Any value of a threshold, limit, duration, etc. provided herein is not intended to be absolute and, thereby, can be approximate. In addition, any threshold, limit, duration, etc. provided herein can be fixed or varied either automatically or by a user. Furthermore, as is used herein relative terminology such as exceeds, greater than, less than, etc. in relation to a reference value is intended to also encompass being equal to the reference value. For example, exceeding a reference value that is positive can encompass being equal to or greater than the reference value. In addition, as is used herein relative terminology such as exceeds, greater than, less than, etc. in relation to a reference value is intended to also encompass an inverse of the disclosed relationship, such as below, less than, greater than, etc. in relations to the reference value. Moreover, although blocks of the various processes may be described in terms of determining whether a value meets or does not meet a particular threshold, the blocks can be similarly understood, for example, in terms of a value (i) being below or above a threshold or (ii) satisfying or not satisfying a threshold.

Features, materials, characteristics, or groups described in conjunction with a particular aspect, embodiment, or example are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

While certain embodiments have been described, these embodiments have been presented by way of example only, and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some embodiments, the actual steps taken in the processes illustrated and/or disclosed may differ from those shown in the figures. Depending on the embodiment, certain of the steps described above may be removed, others may be added. For example, the actual steps and/or order of steps taken in the disclosed processes may differ from those shown in the figure. Depending on the embodiment, certain of the steps described above may be removed, others may be added. For instance, the various components illustrated in the figures may be implemented as software and/or firmware on a processor, controller, ASIC, FPGA, and/or dedicated hardware. Hardware components, such as processors, ASICs, FPGAs, and the like, can include logic circuitry. Furthermore, the features and attributes of the specific embodiments disclosed above may be combined in different ways to form additional embodiments, all of which fall within the scope of the present disclosure.

User interface screens illustrated and described herein can include additional and/or alternative components. These components can include menus, lists, buttons, text boxes, labels, radio buttons, scroll bars, sliders, checkboxes, combo boxes, status bars, dialog boxes, windows, and the like. User interface screens can include additional and/or alternative information. Components can be arranged, grouped, displayed in any suitable order.

Although the present disclosure includes certain embodiments, examples and applications, it will be understood by those skilled in the art that the present disclosure extends beyond the specifically disclosed embodiments to other alternative embodiments and/or uses and obvious modifications and equivalents thereof, including embodiments which do not provide all of the features and advantages set forth herein. Accordingly, the scope of the present disclosure is not intended to be limited by the specific disclosures of preferred embodiments herein, and may be defined by claims as presented herein or as presented in the future.

Conditional language, such as “can,” “could,” “might,” or “may,” unless specifically stated otherwise, or otherwise understood within the context as used, is generally intended to convey that certain embodiments include, while other embodiments do not include, certain features, elements, or steps. Thus, such conditional language is not generally intended to imply that features, elements, or steps are in any way required for one or more embodiments or that one or more embodiments necessarily include logic for deciding, with or without user input or prompting, whether these features, elements, and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. An apparatus for applying negative pressure to a wound, the apparatus comprising:
a negative pressure source configured to provide negative pressure via a fluid flow path to a wound covered by a wound dressing; and
a controller coupled to a memory, the controller configured to:
operate the negative pressure source to provide negative pressure to the wound;
communicate with a remote computing device via a computer network according to a security protocol, wherein the security protocol comprises periodically assigning a new IP address to the apparatus; and
process data received from the remote computing device according to a security rule.

2. The apparatus of claim 1, wherein:
the security protocol further comprises assigning a new IP address to the apparatus for each communication request to the remote computing device and encrypting communications with the remote computing device through mutual authentication; and
the security rule comprises not responding to any redirect requests to a network address different from a network address of the remote computing device.

3. The apparatus of claim 2, wherein the mutual authentication is performed via security certificates stored in the memory of the apparatus and on the remote computing device, and wherein the security certificate stored in the memory uniquely identifies the apparatus.

4. The apparatus of claim 1, wherein:
the memory stores instructions that, when executed by the controller, cause the controller to operate the negative pressure source, communicate with the remote computing device, and process data received from the remote computing device; and
the security rule comprises, in response to receiving from the computing device an update of at least some instructions stored m the memory, verifying an identity of an author of the update prior to updating the at least some instructions.

5. The apparatus of claim 1, further comprising one or more anti-tampering mechanisms configured to indicate unauthorized use of the apparatus.

6. The apparatus of claim 1, wherein the controller is configured to process the data according to the security rule so that access to the data provided by the controller via the computer network is limited to one or more authenticated devices.

7. The apparatus of claim 1, wherein the controller is configured to receive the data according to the security rule so that the data is enabled to adjust a first function performable by the controller and prevented from adjusting a second function performable by the controller.

8. The apparatus of claim 1, wherein the security rule comprises one or more of enforcing code signing, enforcing transport encryption, or utilizing mutual authentication.

9. (canceled)

10. The apparatus of claim 8, wherein transport encryption utilizes transport layer security.

11. (canceled)

12. (canceled)

13. The apparatus of claim 1, wherein the data comprises one or more of data indicative of operations of the negative pressure source identification information for a user of the negative pressure source, or patient data for a user of the apparatus.

14. (canceled)

15. A method of operating a negative pressure wound therapy apparatus, the method comprising:
activating a negative pressure source configured to provide negative pressure via a fluid flow path to a wound covered by a wound dressing;
communicating with a remote computing device via a computer network according to a security protocol, wherein the security protocol comprises periodically assigning a new IP address to the apparatus; and
processing data received from the remote computing device according to a security rule,
wherein the method is performed by a controller of the apparatus.

16. The method of claim 15, wherein:
the security protocol further comprises assigning a new IP address to the apparatus for each communication request to the remote computing device and encrypting communications with the remote computing device through mutual authentication; and
the security rule comprises not responding to any redirect requests to a network address different from a network address of the remote computing device.

17. The method of claim 16, wherein the mutual authentication is performed via security certificates stored in a memory of the apparatus and on the remote computing device, and wherein the security certificate stored in the memory uniquely identifies the apparatus.

18. The method of claim 17, wherein:
the memory stores instructions that, when executed by the controller, cause the controller to operate the negative pressure source, communicate with the remote computing device, and process data received from the remote computing device; and
the security rule comprises, in response to receiving from the computing device an update of at least some instructions stored in the memory, verifying an identity of an author of the update prior to updating the at least some instructions.

19. The method of claim 15, wherein the apparatus further comprises one or more anti-tampering mechanisms configured to indicate unauthorized use of the apparatus.

20. The method of claim 15, wherein the controller is configured to process the data according to the security rule so that access to the data provided by the controller via the computer network is limited to one or more authenticated devices.

21. The method of claim 15, wherein the controller is configured to receive the data according to the security rule so that the data is enabled to adjust a first function performable by the controller and prevented from adjusting a second function performable by the controller.

22. The method of claim 15, wherein the security rule comprises one or more of enforcing code signing, enforcing transport encryption, or utilizing mutual authentication.

23. (canceled)

24. The method of claim 22, wherein transport encryption utilizes transport layer security.

25. (canceled)

26. (canceled)

27. The method of claim 15, wherein the data comprises one or more of data indicative of operations of the negative pressure source, identification information for a user of the negative pressure source, or patient data for a user of the apparatus.

28. (canceled)
